(19) United States
(12) Patent Application Publication
(10) Pub. No.: US 2002/0119914 A1
(43) Pub. Date: Aug. 29, 2002

(54) New uses of insulin and pancreatin

(76) Inventors: Deguang Zhu, New York, NY (US); Yao Zhu, New York, NY (US)

Correspondence Address: DEGUANG ZHU, 718 WEST 171ST STREET, APT. 33, NEW YORK, NY, 10032, US

(21) Appl. No.: 09/748,466

(22) Filed: Dec. 26, 2000

(51) Int. Cl. A61K 7/42; A61K 38/28; A61K 38/00

(52) U.S. Cl. 514/3; 424/59; 514/4; 514/12

(57) ABSTRACT

The present invention discloses new uses of insulin and pancreatin. Insulin and pancreatin can topically be applied to skin, scalp and hair to effectively treat and prevent skin or scalp from aging. Topical application of insulin can prevent skin and scalp from damages caused by oxygen free radicals and UV, promote wound healing, treat and prevent skin from complications caused by diabetes or by topical application of corticosteroids.

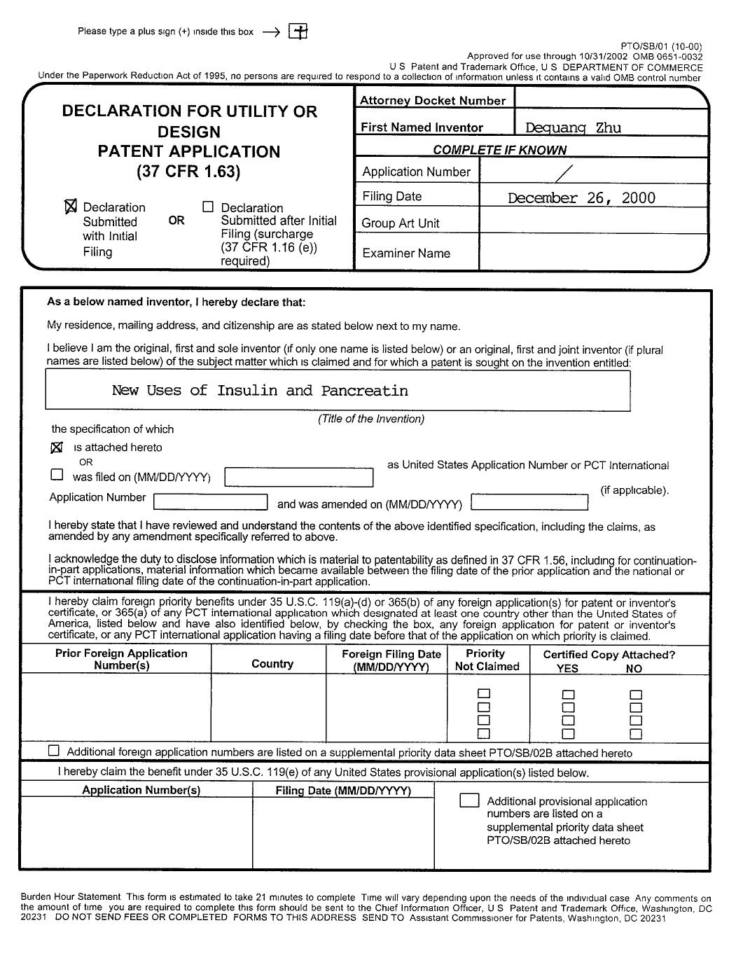

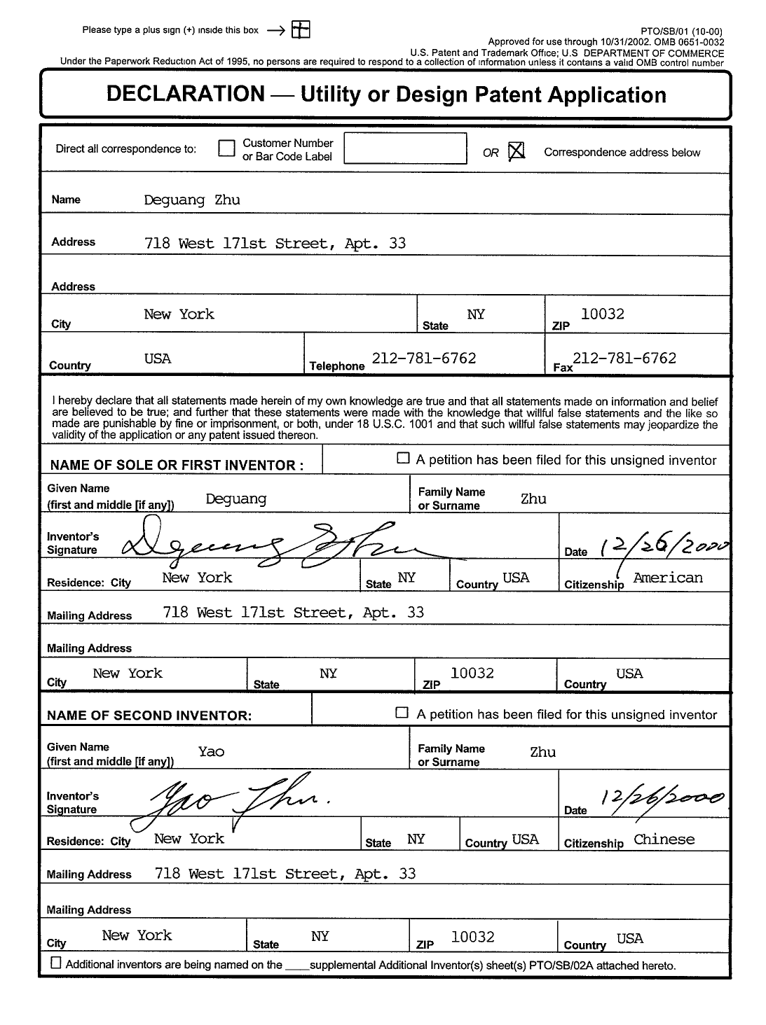

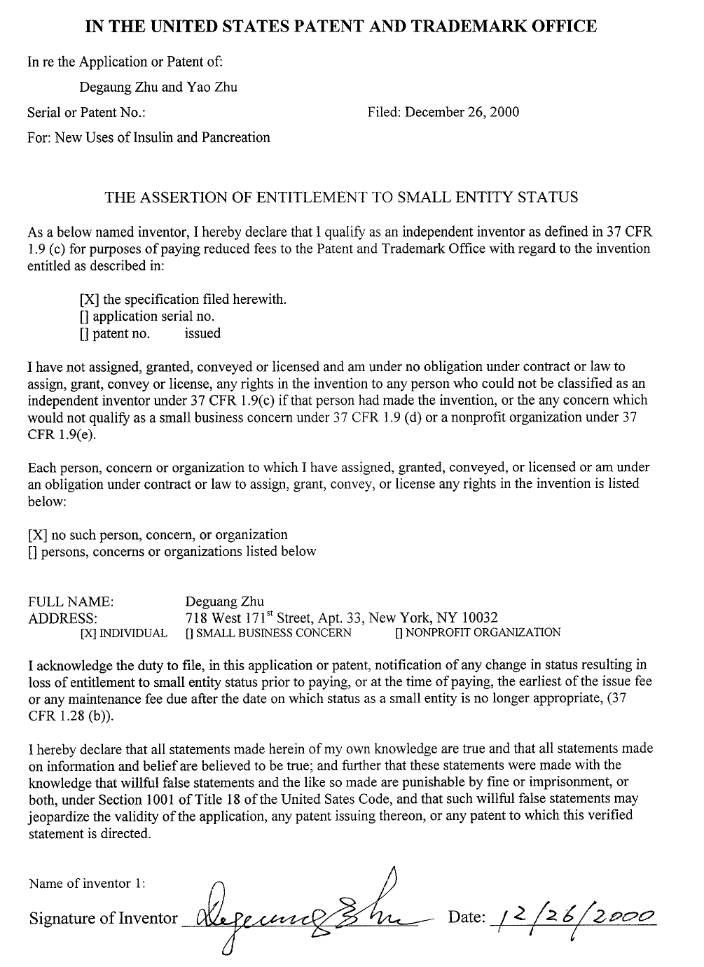

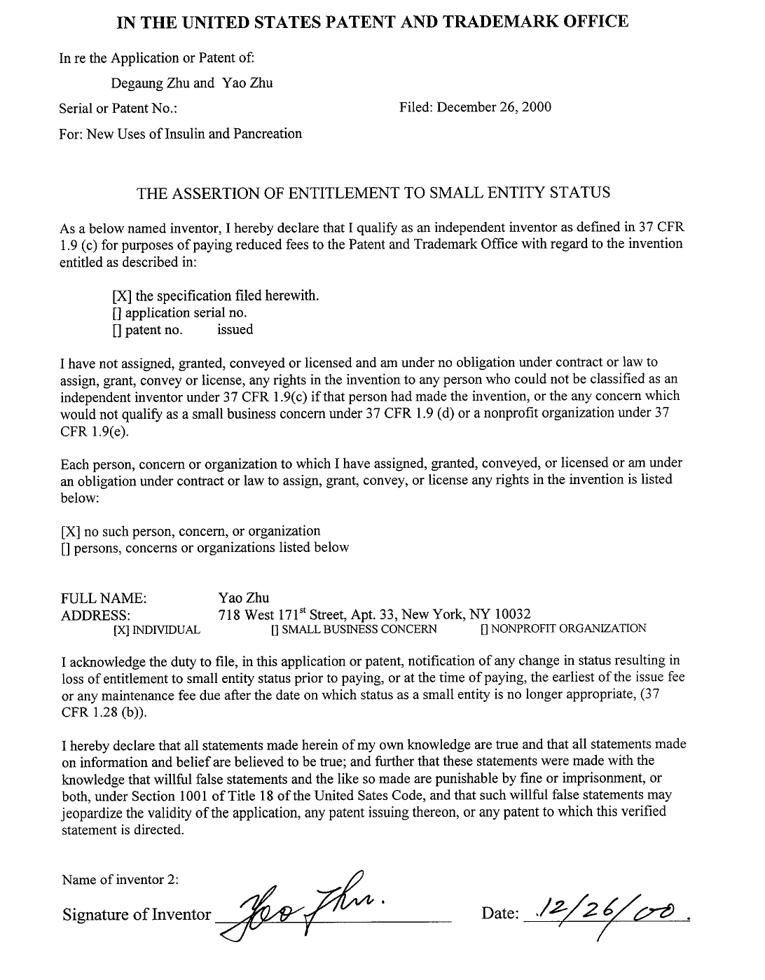

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

Insulin is generally used to treat diabetes, and pancreatin is used as a digestive aid. Both of them are not topically applied to skin to treat skin aging or diseases. In our published international patent application (PCT/US98/21794), we disclosed crude extract of animal pancreas could topically applied to promote skin wound healing as well as to peel stratum corneum. The crude extract of pancreas contains less insulin but a lot of pancreatin. Insulin was disclosed in Lindenbaum's patents (U.S. Pat. Nos. 5,461,030 and 5,591,709) to be able to promote wound healing. However, the pancreatin is digestive enzymes. Theoretically it can digest skin cells. But why can the crude extract of pancreas containing so many digestive enzymes promote skin wound healing but does not make the skin wound worse? We could not understand in PCT/US98/21794. Additionally, effect of individual active composition in the crude extract of animal pancreas on skin was not yet studied respectively in detail. The present invention provides new uses of insulin and pancreatin in cosmetic and dermatological fields, and discloses working mechanism of pancreatin on skin desquamation.

### BRIEF SUMMARY OF THE INVENTION

In an attempt to understand the working mechanism of the crude extract of animal pancreas on skincare, we found insulin could topically be applied to treat and prevent skin from aging and to treat complications of skin and subcutaneous tissue caused by diabetes and by topical application of steroid hormones. Additionally, we found the pancreatin could be used as isoenzyme of skin desquamation enzymes. Like natural desquamation process of skin, the pancreatin can specifically peel excessive stratum corneum but it does not digest other keratinocytes. This probably is the reason that the crude extract of pancreas containing a lot of digestive enzymes is able to promote healing of skin wound.

### BRIEF DESCRIPTION OF THE SEVERAL VIEWS OF THE DRAWING

Not Applicable

### DETAILED DESCRIPTION OF THE INVENTION

New Uses of Insulin

1. Insulin can topically be applied to increase firmness and elasticity of skin and scalp, and reduce lines and wrinkles. Effect of insulin on improving skin firmness is far superior to other anti-aging materials available in current skincare field. Our data indicate 2-week topical treatment of insulin increased skin firmness up to 38% and 4-week treatment up to 45%. This indicates insulin can effectively improve quality of connective tissue, such as collagen, elastin, and proteoglycon.

2. Insulin can topically be applied to promote turnover and proliferation of skin and scalp cells.

3. Insulin can topically be applied to improve age spots and clarity of skin, and make skin more radiance.

4. Insulin can topically be applied to rebuild subcutaneous fat padding.

5. Insulin can topically be applied to improves secretion of sebaceous and sweat glands.

6. Insulin can topically be applied to promote skin and scalp cells to absorb more nutrition.

7. Insulin can topically be applied to raise ability of skin and scalp to scavenge oxygen free radicals.

8. Insulin can topically be applied to treat UV-induced damage and raise ability of skin and scalp against UV-induced damage.

9. Insulin can topically be applied to help skin and scalp to repair damaged keratinocytes.

10. Skin aging includes chronological and photo aging. Chronological aging is a degenerate process of skin and subcutaneous tissues such as basal cells shrink, increase in lines and wrinkles of skin, aging spots, decrease in firmness, elasticity, cell renewal and glands secretion of skin, and subcutaneous fat atrophies. Photoaging is a degenerate process of skin caused by damages of UV and oxygen free radicals. In above new uses 1-6 of insulin, the insulin can effectively restore balance of skin degeneration and regeneration, and in new uses 7-9 of insulin, the insulin can treat, repair and prevent skin or scalp from damages of UV and oxygen free radicals. Therefore, insulin is an excellent anti-aging composition. It can topically be applied to skin and scalp to treat and prevent skin and scalp from aging.

11. Insulin can topically be applied to improve hair growth and quality.

12. Insulin can topically be applied to treat winter itch.

13. Insulin can topically be applied to treat atrophies of local skin.

14. Insulin can topically be applied to promote wound healing.

15. Insulin can topically be applied to treat skin complications caused by topical application of corticosteroids. It can also be formulated in products for topical application containing corticorsteroids to neutralize adverse reactions of corticosteroids to topical skin, scalp, hair, and wound.

16. Insulin can topically be applied to treat or prevent skin and connective tissue from diabetic complications. Therefore, topical application of insulin is a skincare very good for patients with diabetes.

Human and animal insulin has same function. Recombinant and synthetic insulin has the same function as natural insulin. Insulin is compatible with most of cosmetic and pharmaceutical raw materials, and it can be formulated with cosmetically or pharmaceutically acceptable ingredients into products of cosmetic or pharmaceutical. Activity of insulin in skincare products is very stable at room temperature, which can be kept at least for one year. Topical application of insulin to skin is very safe. Insulin does not cause irritation, does not cause adverse reaction, and is not easy to be absorbed by the skin into system. Recommended concentration of insulin in 100 g emulsion or solution can be 2 to 20 Units or more.

### EXAMPLE 1

| | % w/w |
|---|---|
| Water | 86.2 |
| EDTA | 0.1 |
| Carbomer | 0.2 |
| Glycerin | 4.0 |
| Methylparaben | 0.2 |
| Propylparaben | 0.1 |
| Mineral oil | 4.0 |
| Stearic acid | 2.0 |
| Glyceryl strearate | 2.0 |
| Cetyl Alcohol | 0.2 |
| Ceteareth-20 | 0.1 |
| Dimethicone | 0.5 |
| Triethanolamine | 0.4 |
| Insulin | 4 |
| | Unit |

Working Mechanism and New Use of Pancreatin on Skincare

Skin desquamation is a complicated process controlled by enzymes. Hansson and Brattsand isolated respectively stratum corneum chymotryptic and tryptic enzymes (SCCE & SCTE) from stratum corneum, and demonstrated SCCE & SCTE played an important role in human epidermal desquamation process, but shedding process must be in alkaline buffer with chelating agent to catch Ca⁺⁺. Natural skin does not contain chelator. It suggests that more events participate in natural shedding process of skin. Outer layer of stratum corneum consists primarily of proteins and ceramide lipids. The proteins construct a framework and ceramide lipids attach to the proteins by ester bond as cement. SCCE and SCTE are serine proteases. They can digest protein framework of stratum corneum but cannot digest cement of ceramide lipids in outer layer of stratum corneum. Although whole system of skin desquamation enzymes has not been known, we infer that natural desquamation enzymes of skin should include lipases for digestion of ceramide lipids. The pancreatin contains lipases which can digest ceramide lipids, and the pancreatin also contains proteases such as chymotrypsin and trypsin. The chymotrypsin and trypsin also are serine proteases. Chymotrypsin and trypsin not only in structure and function but also in activated passage are similar to SCCE and SCTE. Therefore, the pancreatin and skin desquamation enzymes probably belong in the same family, and the pancreatin can probably be used as isoenzymes of the skin desquamation enzymes. Our experiment demonstrated this hypothesis. Like natural desquamation process of skin, the pancreatin only peels excessive outer layer of stratum corneum but does not peel other keratinocytes. Therefore, the pancreatin is the isoenzymes of skin desquamation enzymes. It can be used to replenish skin desquamation enzymes to restore natural desquamation process of skin. In cosmetic fields, the pancreatin can be used as a highly specific bio-exfoliant to replace nonspecific chemical exfoliants. Compared to chemical exfoliants, the advantage using the pancreatin is the pancreatin can keep intact, healthy keratinocyte layer as well as peel excessive stratum corneum. In dermatological field, the pancreatin can be used to treat dermal diseases with defective desquamation such as ichthyosis, psoriasis, acne, dandruff, and etc. Recommended concentration of pancreatin in 100 g emulsion or solution can be 0.8 g, or more.

Combination of Insulin and Pancreatin

Skin aging involves epidermis, dermis, and subcutaneous tissue. It is characterized by excessive stratum corneum and basal cells shrink in epidermic, decrease of fibroblasts, collagen, elastin, and proteoglycon in dermis, reduction of glands secretion, and subcutaneous fat atrophies. Although the insulin can repair almost all signs of skin degeneration as disclosed in the new uses of insulin, it cannot peel the excessive stratum corneum in skin aging. However, as disclosed above in new use of the pancreatin, the pancreatin is a highly specific bio-exfoliant, it can replenish skin desquamation enzymes, specifically peels excessive stratum corneum but does not damage healthy keratinocytes. Therefore, combination of insulin and pancreatin can treat and prevent skin from aging in every way. Following Example 2 is an example using insulin and pancreatin to make a multi-function anti-aging skincare product.

### EXAMPLE 2

Protective and Repair Cream for Anti-aging Skincare

| | % w/w |
|---|---|
| Water | 70.9 |
| EDTA | 0.1 |
| Carbomer | 0.2 |
| Glycerin | 4.0 |
| Methylparaben | 0.2 |
| Propylparaben | 0.1 |
| Glyceryl stearate | 2.0 |
| Cetyl Alcohol | 0.2 |
| PEG-40 | 0.1 |
| Phenotrimethicone | 1.0 |
| Cyclomethicone | 7.0 |
| Octyl methoxycinnamate | 7.5 |
| Benzophenone-3 | 5.0 |
| Tocopheryl acetate | 0.5 |
| Dimethicone | 0.5 |
| Triethanolamine | 0.5 |
| Perfume | qs |
| Insulin | 2 |
| | Unit |
| Pancreatin | 1.0 |

Scope of the Invention

Although the description above contains many specificities, these should not be construed as limitations on the scope of the invention, but rather as an exemplification of one preferred embodiment thereof. Many other variations are possible. For example, synthetic or recombinant insulin and pancreatin of human or animals, including natural, recombinant or synthetic bioactive materials which have active centers which are the same as or similar to active centers of said insulin and pancreatin, can also offer effects the same as or similar to the effects of natural insulin and pancreatin on skin, scalp, hair and wound. They can also be used as an excellent cosmetic and pharmaceutical composition. Therefore, they should also be included in the scope of the present invention. This is because by means of the current knowledge and technologies on cell molecular biology, it is easy to duplicate artificially any natural proteins or peptides. The function of protein and peptide depends on their active centers. Using synthetic or gene recombination technology, people can change any amino acid in protein or peptide molecule to create an isomer. Said isomer may have the same or similar function as that of natural one if its active centers are not changed or are not changed a lot.

Accordingly, the scope of the invention should be determined not by the embodiments illustrated, but by the appended claims and their legal equivalents.

We claim:

1. A composition for topical application to skin, scalp or hair, which increases skin firmness and elasticity, reduces lines and wrinkles of skin, improves quality of connective tissues, promotes turnover of skin or scalp cells, promotes proliferation of skin or scalp cells, helps skin to repair damaged keratinocytes, improves age stops and clarity of skin, raises ability of skin or scalp to scavenge oxygen free radicals, treats UV-induced damage, raises ability of skin or scalp against UV-induced damage, promotes skin or scalp cells to absorb nutrition, rebuilds subcutaneous fat padding, treats aging of skin or scalp, prevents skin or scalp from aging, improves hair growth and quality, treats winter itch or atrophies of local skin, treats and neutralizes skin complications of corticosteroids in topical application, treats complications of skin and connective tissue caused by diabetes, prevents skin and connective tissue from complications caused by diabetes, and/or improves secretion of sebaceous and sweat glands, comprising:
insulin which can be natural, synthetic, recombinant, human or animal; or
a bioactive material which has active centers which are the same as or similar to active centers of said insulin.

2. The composition for topical application to the skin, scalp and hair according to claim 1, wherein said composition further includes cosmetically or pharmaceutically acceptable ingredients to formulate a product of cosmetic or pharmaceutical.

3. The composition for topical application to the skin or scalp according to claim 1, wherein said composition comprising a material, which treats skin or scalp aging or prevents skin or scalp from aging, and which is said insulin or is said bioactive material which has said active centers which are the same as or similar to said active centers of said insulin.

4. A composition for topical application to skin or scalp, which replenishes skin desquamation enzymes, specifically peels excessive stratum corneum, treats aging of skin or scalp, prevents skin or scalp from aging, or treats dermal diseases with defective desquamation such as ichthyosis, psoriasis, acne, dandruff, and etc, comprising:
pancreatin which can be natural, synthetic, recombinant, human or animal; or
a bioactive material which has active centers which are the same as or similar to active centers of said pancreatin.

5. A method of increasing skin firmness and elasticity, reducing lines and wrinkles of skin, improving quality of connective tissues, promoting turnover of skin or scalp cells, promoting proliferation of skin or scalp cells, helping skin or scalp to repair damaged keratinocytes, improving age stops and clarity of skin, raising ability of skin or scalp to scavenge oxygen free radicals, treating UV-induced damage, raising ability of skin or scalp against UV-induced damage, promoting skin or scalp cells to absorb nutrition, rebuilding subcutaneous fat padding, treating aging of skin or scalp, preventing skin or scalp from aging, improving hair growth and quality, treating winter itch or atrophies of local skin, treating and neutralizing skin complications of corticosteroids in topical application, treating complications of skin or connective tissue caused by diabetes, preventing skin and connective tissue from complications caused by diabetes, or improving secretion of sebaceous and sweat glands comprising:
topically applying insulin, which can be natural, synthetic, recombinant, human or animal, to skin, scalp or hair; or
topically applying a bioactive material, which has active centers which are the same as or similar to active centers of said insulin, to skin, scalp or hair.

6. The method of claim 5, wherein said method further includes using cosmetically or pharmaceutically acceptable ingredients to formulate a product of cosmetic or pharmaceutical comprising:
said insulin which can be natural, synthetic, recombinant, human or animal; or
said bioactive material which has said active centers which are the same as or similar to said active centers of said insulin.

7. The method of claim 5, wherein said method of treating skin aging or protecting skin from aging includes the topical application to the skin of a composition comprising:
said insulin which can be natural, synthetic, recombinant, human or animal; or
said bioactive material which has said active centers which are the same as or similar to said active centers of said insulin.
